(19) United States
(12) Patent Application Publication
(10) Pub. No.: US 2008/0069827 A1
(43) Pub. Date: Mar. 20, 2008

(54) miRNA of HSV-LAT and uses thereof

(76) Inventors: Nigel Fraser, Merion Station, PA (US); Ananya Gupta, Philadelphia, PA (US)

Correspondence Address: PEARL COHEN ZEDEK LATZER, LLP, 1500 BROADWAY 12TH FLOOR, NEW YORK, NY, 10036, US

(21) Appl. No.: 11/889,220

(22) Filed: Aug. 9, 2007

Related U.S. Application Data
(60) Provisional application No. 60/836,385, filed on Aug. 9, 2006.

(51) Int. Cl. A61K 31/70; A61K 39/00; A61P 43/00; C12Q 1/02; C12Q 1/68

(52) U.S. Cl. 424/184.1; 435/29; 435/6; 514/44

(57) ABSTRACT

This invention relates to treatment of Herpes Symplex Virus infection. Specifically, the invention relates to the use of a newly discovered miRNA in the silencing of HSV-LAT gene, which is responsible for the prevention of apoptosis of infected neuronal cells and the maintenance of latency.

### CROSS REFERENCE TO RELATED APPLICATIONS

This application claims priority from U.S. Provisional Application Ser. No. 60/836,385 filed Aug. 9, 2006, which is incorporated herein by reference in its entirety.

### FIELD OF INVENTION

This invention is directed to treatment of Herpes Symplex Virus infection. Specifically, the invention relates to the use of a newly discovered miRNA in the silencing of HSV-LAT gene, which is responsible for inhibiting apoptosis of infected neuronal cells and the maintenance of latency.

### BACKGROUND OF THE INVENTION

Herpes simplex viruses (HSV) are human herpes viruses of the neurotropic alpha herpes virus subfamily. Infections with HSV type 1 (HSV-1) and HSV type 2 (HSV-2) are highly prevalent. The usual manifestations of disease are mucocutaneous lesions of the mouth, face, eyes, or genitalia. Both HSV-1 and HSV-2 establish latent infections in neurons of peripheral ganglia and may reactivate to cause recurrent lesions. Rarely, the virus spreads to the central nervous system to cause meningitis or encephalitis. Generalized or disseminated HSV infection may occur in patients immunologically compromised by neoplasia, organ transplantation, inherited immunodeficiency disease, or AIDS, or through neonatal infection acquired by transmission of the virus through an infected birth canal.

Herpes simplex virus (HSV) is a prevalent cause of genital infection in humans, with an estimated annual incidence of 600,000 new cases and with 10 to 20 million individuals experiencing symptomatic chronic recurrent disease. The asymptomatic subclinical infection rate may be even higher.

MicroRNAs (miRNAs) are a class of small RNA molecules that regulate the stability or the translational efficiency of target messenger RNAs (mRNAs). The latency associated transcript (LAT) of Herpes simplex virus-1 (HSV-1) is the only viral gene expressed during latent infection in neurons. LAT inhibits apoptosis, and maintains latency by promoting the survival of infected neurons.

Although continuous administration of antiviral drugs such as acyclovir ameliorates the severity of acute HSV disease and reduces the frequency and duration of recurrent episodes, such intervention does not abrogate the establishment of latency nor does it alter the status of the latent virus. As a consequence, the recurrent disease pattern is rapidly reestablished upon cessation of treatment. Since the main source of virus transmission arises from recrudescence, an approach to impact the rate of infection is required. Thus, it is a matter of great medical and scientific interest to provide safe and effective agents for humans to prevent HSV infection, and recrudescence.

### SUMMARY OF THE INVENTION

In one embodiment, provided herein is a method of treating Herpes Simplex Virus (HSV) infection in a subject, comprising administering to said subject an agent capable of inhibiting the function of a HSV-latency-associated transcript (LAT) gene in said subject, whereby said latency-associated gene inhibits apoptosis of infected neurons.

In another embodiment, provided herein is a composition for the treatment of Herpes Simplex Virus infection in a subject, comprising an agent capable of inhibiting the function of a HSV latency-associated transcript (LAT) gene.

In one embodiment, provided herein is a method for inhibiting apoptosis of neuronal cells in a subject, comprising contacting said neuronal cells with a miRNA of HSV-LAT gene, wherein said miRNA modulates the TGF-b pathway, thereby inhibiting apoptosis of said neuronal cells.

In another embodiment, provided herein is a method of screening for therapeutic agents for the treatment of HSV infection in a subject, comprising the step of: contacting a neuronal cell of said subject with the candidate therapeutic agent; and analyzing for the function of HSV-LAT gene or its encoded proteins in said contacted cell, wherein inhibition of the function of HSV-LAT gene or its encoded proteins in said neuronal cell indicates the candidate therapeutic agent is effective in treating HSV infection.

In one embodiment, provided herein is a vaccine for treating, preventing or ameliorating a subject against herpes simplex virus infection or recrudescence, comprising a pharmaceutically acceptable carrier and an effective amount of an agent capable of inhibiting the function of a HSV-latency-associated transcript (LAT) gene in said subject, thereby allowing for apoptosis of infected neurons.

### BRIEF DESCRIPTION OF THE DRAWINGS

FIG. 1. shows that LAT inhibits apoptosis by a Dicer dependent mechanism. (a) RT-PCR analysis of the transcript levels of Dicer and GAPDH after transfection of SY5Y cells with siRNA for Dicer. (b) Western blot analysis for Dicer and β-actin protein using whole cell lysates of SY5Y cells transfected with siRNA against Dicer. (C) SY5Y cells were transfected with indicated plasmids, treated with cisplatin and the percentage apoptosis was determined. Data represents the mean of three independent experiments±s.d.

FIG. 2 shows that the LAT gene of HSV codes for a miRNA. (a) Schematic representation of HSV-1 LAT gene. Sequence of mature miRNA is shown in red. Lower panel: Sequence conservation of mirLAT encoding region in 17+, F and McKrae strains of HSV. (b) SY5Y cells were treated with cisplatin 24 h post transfection and the percentage apoptosis was determined. Data represents the mean of three independent experiments±s.d. (c) Pre-mirLAT is processed invitro by recombinant Dicer. (d) Northern blot analysis of the LAT miRNA in SY5Y cells transfected with pcDNA or pcDNA-PstMlu. (e) Northern blot detection of LAT miRNA in cells infected with indicated viruses. M, mock infected.

FIG. 3 shows the LAT region of HSV 17+ protects cells from apoptosis. (a) SY5Y and (b) HeLa cells were infected as indicated and treated with cisplatin 16 hours post infection. Data represents the mean of three independent experiments±s.d. (c) SY5Y and (d) HeLa cells were transfected as indicated, treated with cisplatin 24 h post transfection, and percentage apoptosis was determined. Data represents the mean of three independent experiments±s.d. (e) SY5Y and (f) HeLa cells were treated with cisplatin, 24 h post transfection with indicated miRNA and the percentage apoptosis was determined. Data represents the mean of three independent experiments±s.d. FIG. 4 shows the Modulation of TGF-β signaling by mirLAT. (a) The predicted duplex of mirLAT and its target site in 3′UTR of TGF-β (Upper panel) or in 3′UTR of SMAD-3 (Lower Panel); canonical base-pairs are marked with red circles, mismatches are shown as black circles. Middle panel: The sequence alignments show nucleotide conservation of the mirLAT binding site within TGF-β-UTR. (b) Upper panel: RT-PCR analysis of the transcript levels of TGF-β, SMAD-3, TGF-β receptor I and GAPDH. Lower panel: Western blot analysis for SMAD-3, TGF-β receptor I and β-actin. (c) Upper panel: SY5Y cells were co-transfected with indicated renilla luciferase (RL) constructs along with the pSV β-gal (β-gal) in the absence or presence of mirLAT. Data represents the mean of normalized RL/β-gal activities of four independent experiments±s.d. Lower panel: Transcript levels of RL and FL (as a normalization control) mRNAs as determined by RT-PCR. (d) SY5Y cells were transfected as indicated. Data represents the mean of normalized RL/β-gal activities of four independent experiments±s.d. (e) Upper panel: SY5Y cells were transfected as indicated. Data represents the mean of normalized RL/β-gal activities of four independent experiments±s.d. Lower panel: Transcript levels of RL and FL (as a normalization control) as determined by RT-PCR. (f) Upper panel: SY5Y cells were treated with cisplatin, 24 h post transfection, and the percentage apoptosis was determined. Data represents the mean of four independent experiments±s.d. Lower panel: RT-PCR analysis of the transcript levels of TGF-β, SMAD3 and GAPDH. (g,h,i) SY5Y cells were transfected as indicated along with SBE4-Luc reporter gene. Cells were treated with or without recombinant human TGF-β and analyzed for luciferase activity. Data represents the mean of four independent experiments±s.d.

FIG. 5 shows the inhibition of apoptosis by the HSV-1 LAT gene. (a) SY5Y cells were transiently transfected with (1 μg) pcDNA-PstMlu (a plasmid expressing a fragment of the HSV-1 LAT gene) (black bars) or (1 μg) pcDNA empty vector (white bars). Cells were treated with cisplatin (10 μM) or with etoposide (1 μM), 24 h post transfection and percentage apoptosis was determined as mentioned in methods. Data represents the mean of three independent experiments±s.d. (b) HeLa cells were transfected as described in (a) and treated with cisplatin (10 μM) or etoposide (100 μM). The percentage apoptosis was determined as mentioned in the methods. Data represents the mean of three independent experiments±s.d.

FIG. 6 shows how siRNA against Dicer inhibits RNA silencing. SY5Y cells were transfected with pEGFP and pSilencer-GFP-shRNA in the presence or absence of control siRNA or a siRNA directed against Dicer. GFP expressing cells were visualized and photographed using a fluorescence microscope (×20).

FIG. 7 shows that LAT region of HSV 17+ protects cells from apoptosis. SY5Y cells were mock infected or infected with 17+, ΔSty, and StyR viruses, then treated with cisplatin (10 μM). Representative fields of control (untreated) and cisplatin treated SY5Y cells stained with AnnexinV-FITC (green) are shown. Cells were visualized by fluorescence microscopy (×20).

FIG. 8 shows how an antisense Oligo specific for mirLAT inhibits its effect on apoptosis SY5Y cells were transfected with indicated plasmids and oligos, treated with cisplatin 24 h post transfection and stained with AnnexinV-FITC (green). Cells were visualized by fluorescence microscopy (×20).

FIG. 9 shows how mirLAT protects cells from apoptosis SY5Y cells transiently transfected with dsRNA mirLAT or control miRNA. Representative fields of control (untreated) and cisplatin treated SY5Y cells stained with AnnexinV-FITC (green) are shown. Cells were visualized and photographed using a fluorescence microscope (×20).

FIG. 10 shows the inhibition of TGF-β production by the LAT-expressing cells. Frequency of cells producing latent TGF-β was determined after transient transfection of SY5Y cells with pcDNA-PstMlu (a plasmid expressing a fragment of the HSV-1 LAT gene) or pcDNA empty vector; and mock infection or infection with wild type 17+ Virus; according to the manufacturer's instructions using a Human Latent TGF-β 1 ELISpot Kit (R&D Systems). Results shown are average values obtained from a representative experiment done in triplicates.

FIG. 11 shows sequence of duplex oligonucleotides comprising the predicted wild type and mutated mirLAT response elements. The sequence of the mirLAT-binding site is shown in bold. The mutant bases are underlined. Double stranded annealed oligos (IDT), containing the wild type and mutated sites were cloned in the NdeI-XhoI site of the PRL-TK vector. The identity of the constructs was confirmed by sequencing.

FIG. 12 shows miR-LAT is expressed in F (weakly virulent) and strain McKrae (strongly virulent) of HSV-1 strain.

FIG. 13 shows that miR-LAT expression of TGFb and SMAD-3 is regulated by infections by HSV-1 strain 17, F, McKrae.

FIG. 14 shows the prevalence of miR-LAT in HSV-1 infected cells

### DETAILED DESCRIPTION OF THE INVENTION

This invention relates in one embodiment to treatment of Herpes Symplex Virus infection. In another embodiment, described herein is the use of a newly discovered miRNA in the silencing of HSV-LAT gene, which is responsible for inhibiting apoptosis of infected peripheral neuron cells and the maintenance of latency.

In one embodiment, herpes virus infections are able to persist in the host for long periods in a non-replicative or latent state. Herpes simplex virus type 1 (HSV-1) establishes latent infection in human peripheral sensory ganglia and can reactivate to produce recurrent mucocutaneous lesions. Pathogenesis of herpes virus infections can be divided in one embodiment, into several distinct stages: acute viral replication, establishment of latency, maintenance, and reactivation. Following infection, HSV-1 replicates at the site of infection and is transported to sensory ganglia. Replication at the periphery or in sensory ganglia may increase the amount of virus that can establish latent infection. During latent infection, HSV-1 DNA can be detected in infected tissues but infectious virus cannot be detected. This latent state is maintained in another embodiment, for the life of the host. A variety of stimulae (such as fibrile illness and exposure to ultraviolet irradiation in certain embodiments) can interrupt the latent state and cause the reappearance of infectious virus or reactivation.

In one embodiment, the virus does not produce any detectable protein product during latency, however, there is continuing RNA transcription. This latency associated transcription comes in another embodiment, from a single region of the viral genome (the latency associated transcript or LAT region) and is driven by the latency active promoter (LAP). The TATA box and basal transcriptional regulatory sequences, which constitute the core LAT promoter, reside approximately 700 bp upstream of the 2 kb major LAT

In one embodiment, the HSV-1 LAT gene (position +415 to +475) codes for a miRNA, that inhibits apoptosis. The LAT gene maintains latent infections by protecting infected neurons from undergoing apoptosis. In another embodiment, an unrelated anti-apoptotic gene can substitute for the HSV LAT gene during reactivation of the virus in mice. In one embodiment, the anti-apoptotic miRNA, mirLAT, encoded by the LAT region of HSV-1, plays an important role in the survival of latently infected neurons and thus contributes to the persistence of infection. In one embodiment, mutations in the HSV-LAT gene will affect its ability to establish prolonged latency in the infected cell.

In another embodiment, the anti-apoptotic miRNA, mirLAT, encoded by the LAT region of HSV-1, plays an important role in the survival of latently infected neurons and thus contributes to the persistence of infection and its recrudescence. In one embodiment mammalian cells use RNAi mechanism to restrict viral propagation.

Therefore, according to this aspect of the invention and in one embodiment, provided herein is a method of treating Herpes Simplex Virus (HSV) infection in a subject, comprising administering to said subject an agent capable of inhibiting the function of a HSV-latency-associated transcript (LAT) gene in said subject, whereby said latency-associated gene inhibits apoptosis of infected neurons.

In one embodiment, inhibiting the function of a HSV latency-associated transcript (LAT) gene or its encoded proteins, as used in the methods, compositions and vaccines described herein; comprises lowering the level of a protein or a nucleic acid regulating the function of said latency-associated transcript (LAT) gene, or its encoded proteins. Lowering the level of HSV-LAT refers in one embodiment to a reduction in observed activity of infected cells under circumstances known to induce viral activity had the HSV-LAT been fully functional. These circumstances include in another embodiment, the onset of viral DNA replication and the like. In one embodiment, reduction in HSV-LAT function as used herein, may refer to induction of apoptosis of peripheral neurons, which under circumstances where had the HSV-LAT functionality not been impaired, would not have undergone apoptosis. These circumstances may involve in yet another embodiment, the observation of concentration of TGF-β1 and SMAD3, comparable to uninfected peripheral neurons.

The mechanism by which RNAi functions, is based in one embodiment on the ability of double stranded RNA to induce the degradation of specific RNA molecules. This mechanism involves the conversion of double-stranded RNA into short RNAs that direct ribonucleases to homologous RNA targets (e.g., mRNA targets). In one embodiment, the interference process occurs posttranscriptionally and involves mRNA degradation.

In one embodiment, the nucleic acid regulating the function of said HSV latency-associated transcript (LAT) gene, is a miRNA of said gene. The term “nucleic acid” refers to deoxyribonucleotides or ribonucleotides and polymers thereof in either single- or double-stranded form. The term encompasses in other embodiments; nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Such analogs include in other embodiments, phosphorothioates, phosphoramidates, methyl phosphonates, chiral methyl phosphonates, 2-O-methyl ribonucleotides, and peptide-nucleic acids (PNAs).

The term “miRNA” refers in one embodiment, to microRNA, a class of small RNA molecules or a small noncoding RNA molecules, that are capable of causing interference, inhibition of RNA translation into protein, and can cause post-transcriptional silencing of specific genes in cells. In one embodiment, miRNAs refers to small temporal RNAs (stRNAs), which belong to a class of non-coding microRNAs, shown in another embodiment to control gene expression either by repressing translation or by degrading the targeted mRNAs that are generally 20-28 nt (nucleotides) in length, such as the miRNA represented by the nt sequence GUGGCGGCCC GGCCCGGGGCCTT (SEQ ID NO. 1), having 23 nt or the miRNA represented by the nt sequence UGGCGGCCCG GCCCGGGGCC (SEQ ID NO. 6), having 20 nt. In one embodiment, miRNAs or stRNAs are not encoded by any microgenes, but are generated from aberrant, dsRNAs by an enzyme called Dicer, which cuts double-stranded RNA into little pieces. In one embodiment, the miRNA that regulates the function of HSV-LAT comprises the nucleotide sequence of SEQ ID NO. 1, SEQ ID No. 5 or SEQ ID NO. 6.

In one embodiment, the agent capable of inhibiting the function of a HSV-latency-associated transcript (LAT) gene, which is used in the methods, compositions and vaccines described herein, is a siRNA, polyamides, triple-helix-forming agents, antisense RNA, synthetic peptide nucleic acids (PNAs), agRNA, LNA/DNA copolymers, small molecule chemical compounds, or a combination thereof.

In one embodiment, the term “siRNA” refers to RNA interference, which in another embodiment refers to the process of sequence-specific post-transcriptional gene silencing in animals, mediated by short interfering RNAs (siRNAs). In another embodiment, the process of post-transcriptional gene silencing is an evolutionarily-conserved cellular defense mechanism used to prevent the expression of foreign genes. Such protection from foreign gene expression evolved in one embodiment, in response to the production of double-stranded RNAs (dsRNAs) derived from viral infection or in another embodiment, from the random integration of transposon elements into a host genome via a cellular response that specifically destroys homologous single-stranded RNA of viral genomic RNA. In one embodiment, the presence of dsRNA in cells triggers the RNAi response.

In one embodiment, the term “conserved”, refers to amino acid sequences comprising the peptides or nucleotides described herein, which remain in one embodiment, essentially unchanged throughout evolution, and exhibit homology among various species producing the protein.

The presence of long dsRNAs in cells stimulates in another embodiment, the activity of a ribonuclease III enzyme referred to as dicer. Dicer is involved in one embodiment, in the processing of the dsRNA into short pieces of dsRNA known as short interfering RNAs (siRNAs). Short interfering RNAs derived from dicer activity are in another embodiment about 21 to about 23 nucleotides in length and comprise about 19 base pair duplexes. Small RNAs function in one embodiment, by base-pairing to complementary RNA or DNA target sequences. When bound to RNA, small RNAs trigger RNA cleavage in another embodiment, or translational inhibition of the target sequence in another embodiment. When bound to DNA target sequences, small interfering RNAs mediate in one embodiment, DNA methylation of the target sequence. The consequence of these events, in one embodiment, is the inhibition of gene expression, which, in another embodiment is the HSV-LAT gene described herein. In one embodiment, the agent used for reducing the function of HSV-LAT gene or its encoded protein, is a siRNA specific for the nucleic acide encoding HSV-LAT.

In one embodiment, the siRNA of the HSV-LATgene described herein, exhibit substantial complimentarity to its target sequence. In another embodiment, “complementarity” indicates that the oligonucleotide has a base sequence containing an at least 15 contiguous base region that is at least 70% complementary, or in another embodiment at least 80% complementary, or in another embodiment at least 90% complementary, or in another embodiment 100% complementary to an-at least 15 contiguous base region present of a target gene sequence (excluding RNA and DNA equivalents). (Those skilled in the art will readily appreciate modifications that could be made to the hybridization assay conditions at various percentages of complementarity to permit hybridization of the oligonucleotide to the target sequence while preventing unacceptable levels of non-specific hybridization). The degree of complementarity is determined by comparing the order of nucleobases making up the two sequences and does not take into consideration other structural differences which may exist between the two sequences, provided the structural differences do not prevent hydrogen bonding with complementary bases. The degree of complementarity between two sequences can also be expressed in terms of the number of base mismatches present in each set of at least 15 contiguous bases being compared, which may range from 0-3 base mismatches, so long as their functionality for the purpose used is not compromised.

In one embodiment, the siRNA of the HSV-LAT gene described herein is sufficiently complimentary to its target sequence. “Sufficiently complementary” refers in one embodiment to a contiguous nucleic acid base sequence that is capable of hybridizing to another base sequence by hydrogen bonding between a series of complementary bases. In another embodiment, complementary base sequences may be complementary at each position in the base sequence of an oligonucleotide using standard base pairing (e.g., G:C, A:T or A:U pairing) or may contain one or more residues that are not complementary using standard hydrogen bonding (including abasic “nucleotides”), but in which the entire complementary base sequence is capable of specifically hybridizing with another base sequence under appropriate hybridization conditions. Contiguous bases are at least about 80% in one embodiment, or at least about 90% in another embodiment, or about 100% complementary to a sequence to which an oligonucleotide is intended to specifically hybridize in another embodiment. Appropriate hybridization conditions are well known to those skilled in the art, can be predicted readily based on base sequence composition, or can be determined empirically by using routine testing (e.g., See Sambrook et al., Molecular Cloning. A Laboratory Manual, 2^{nd} ed. (Cold Spring Harbor Laboratory Press, Cold Spring Harbor, N.Y., 1989).

In one embodiment, minor groove-binding N-methyl pyrrole (Py) and N-methylimidazole (Im) polyamides (peptides) uniquely recognize each of the four Watson-Crick base pairs. Antiparallel pairing of imidazole with pyrrole (Im/Py) recognizes in opne embodiment, a G-C base pair, whereas in another embodiment, a Py/Py pair recognizes either an A-T or T-A base pair. The binding constant and sequence-specificity of the Py-Im hairpin polyamides are similar to that of a transcription factor. Therefore, many genes, are silenced in other embodiments, by competitive binding of Py-Im hairpin polyamides to their regulatory sequences. Gene expression is controlled in one embodiment, by a combination of multiple common trascription factors. Inhibition of gene expression through the binding of Py-Im polyamides to regulatory sequences is unique to a specific gene, and contains part of the recognition sequence of the transcription factor together with the unique flanking sequences. In another embodiment, targeting Py-Im polyamide to the coding region is more straightforward when selecting a unique sequence. In one embodiment, the agent used to silence the HSV-LAT gene in the methods, vaccines and compositions described herein, is Py-Im polyamide specific for the miRNA region of HSV-LAT that comprises SEQ ID NO. 1, SEQ ID No. 5 or SEQ ID NO. 6, or to regulatory sequences is unique to HSV-LAT in another embodiment. In another embodiment, the agent used to silence the HSV-LAT gene in the methods and compositions described herein, is a synthetic polyamide nucleic acid (PNA) specific for the coding region of HSV-LAT, or to regulatory sequences, which is unique to HSV-LA Tin another embodiment, such as the miRNA sequences of SEQ ID NO. 1, SEQ ID No. 5 or SEQ ID NO. 6.

In one embodiment, the polyamides used in the compositions vaccines and methods described herein, which, in another embodiment are referred to as “peptide nucleic acid” (PNA) or “synthetic peptide nucleic acids”, is an alkylating Py-Im polyamides that show sequence-specific DNA alkylation. In another embodiment, alkylation of a template strand in the miRNA region comprising SEQ ID NO. 1, SEQ ID No. 5 or SEQ I) NO. 6 of HSV-LAT, by Py-Im polyamide-cyclopropylpyrroloindole (CPI) conjugates with a vinyl linker results in the production of truncated mRNA, effectively inhibiting transcription of HSV-LAT in vitro. In one embodiment, Py-Im tetra-hydro-cyclo-propabenzindolone (CBI) conjugates with indole linkers are the alkylating polyamides used as the agent capable of inhibiting the expression or function of HSV-LAT gene, because indole-CBI has increased chemical stability under acidic and basic conditions.

In one embodiment, oligodeoxynucleotides inhibit cellular transcription by binding to duplex DNA to form a triple helix. Due to the possibility of long-term inhibition of the gene product, oligodeoxynucleotides that can bind duplex DNA have advantages over those that bind mRNA or proteins. These oligodeoxynucleotides are generally called triplex forming oligonucleotides (TFOs). By using DNA-specific TFOs, the inhibition of expression of several cellular genes has been demonstrated, including the oncogene, c-myc, the human immunodeficiency virus-1, the alpha chain of the interleukin 2 receptor, the epidermal growth factor receptor, the progesterone responsive gene and the mouse insulin receptor. In one embodiment, the oligonucleotides used in the methods and compositions described herein, can bind to duplex DNA and form triple helices in a sequence-specific manner and will silence expression or function of HSV-LAT.

In one embodiment, homopyrimidine DNA strand (triplex forming oligonucleotide, TFO) can bind to a homopurine/homopyrimide DNA duplex in the major groove by forming Hoogsteen base pairs with the homopurine strand. The Hoogsteen base pairing scheme mediates sequence specific recognition of the double stranded DNA by the TFO where in one embodiment, an AT base pair is recognized by a T; and a GC base pair by a C that is protonated at N3⁺. In another embodiment, homopurine strands specifically form a DNA triplex in which the AT base pair is contacted by an A; and the GC base pair by a G. In one embodiment, the agent capable of inhibiting the expression or function of HSV-LAT gene is a triple-helix-forming agents. In another embodiment, the triple-helix-forming agents are olygonucletides. In one embodiment, oligonucleotide-mediated triplex formation prevent transcription factor binding to promoter sites and block mRNA synthesis in vitro and in vivo. In another embodiment, DNA intercalating or cross-linking agents are used to prolong oligonucleotide-duplex interactions.

In one embodiment, the term “TFO” or “triplex forming oligonucleotide” refers to the synthetic oligonucleotides of the present invention which are capable of forming a triple helix by binding in the major groove with a duplex DNA structure.

In another embodiment, the tern “bases” refers to both the deoxyribonucleic acids and ribonucleic acids. The following abbreviations are used, “A” refers to adenine as well as to its deoxyribose derivative, “T” refers to thymine, “U” refers to uridine, “G” refers to guanine as well as its deoxyribose derivative, “C” refers to cytosine as well as its deoxyribose derivative. A person having ordinary skill in this art would readily recognize that these bases may be modified or derivatized to optimize the methods described herein, without changing the scope of the invention.

The term “nucleic acid” as used in connection with siRNA, refers in one embodiment to a polymer or oligomer composed of nucleotide units (ribonucleotides, deoxyribonucleotides or related structural variants or synthetic analogs thereof) linked via phosphodiester bonds (or related structural variants or synthetic analogs thereof). Thus, the term refers to a nucleotide polymer in which the nucleotides and the linkages between them are naturally occurring (DNA or RNA), as well as various analogs, for example and without limitation, peptide-nucleic acids (PNAs), phosphoramidates, phosphorothioates, methyl phosphonates, 2-O-methyl ribonucleic acids, and the like. In one embodiment, the siRNAs used in the compositions and methods of the invention, are nucleic acid sequences.

In one embodiment oligomeric antisense compounds, particularly oligonucleotides, are used in modulating the function of nucleic acid molecules of HSV-LAT, ultimately modulating the amount of HSV-LAT miRNA produced. This is accomplished by providing antisense compounds which specifically hybridize with one or more nucleic acids as described in SEQ ID NO.'s 1, 5 and 6. As used herein, the terms “target nucleic acid” and “nucleic acid encoding HSV-LAT, RNA (including pre-mRNA and mRNA) transcribed from such DNA, and also cDNA derived from such RNA. The specific hybridization of an oligomeric compound with its target nucleic acid interferes in another embodiment, with the normal function of the nucleic acid. The modulation of function of a target nucleic acid by compounds which specifically hybridize to it, is referred to in one embodiment as “antisense”. In one embodiment, the functions of DNA to be interfered with using the antisense oligonucleotides described herein, which are used in the methods, vaccines and compositions described herein, include replication and transcription. In another embodiment, functions of RNA to be interfered with include all vital functions such as, for example, translocation of the, RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the function of HSV-LAT. In one embodiment, inhibition of gene expression is preferred and mRNA is a preferred target. In one embodiment, since many genes (including HSV-LAT) have multiple transcripts, “inhibition” also includes an alteration in the ratio between gene products, such as alteration of mRNA splice products.

In one embodiment, specific nucleic acids are targeted for antisense. “Targeting” an antisense compound to a particular nucleic acid, in one embodiment, is a multistep process. The process usually begins with the identification of a nucleic acid sequence whose function is to be inhibited. This may be, for example, a cellular gene (or mRNA transcribed from the gene) whose expression is associated with a particular disorder or disease state, or a nucleic acid molecule from an infectious agent. In one embodiment, the target is a nucleic acid molecule encoding HSV-LAT. The targeting process also includes in another embodiment, determination of a site or sites within this gene for the antisense interaction to occur such that the desired effect, e.g., inhibition of expression of the protein such as menin, will result. In one embodiment, an intragenic site is the region encompassing the translation initiation or termination codon of the open reading frame (ORF) of the gene. Since, the translation initiation codon is in one embodiment 5′-AUG (in transcribed mRNA molecules; 5′-ATG in the corresponding DNA molecule), the translation initiation codon is referred to in one embodiment as the “AUG codon,” the “start codon” or the “AUG start codon”. In another embodiment, a minority of genes have a translation initiation codon having the RNA sequence 5′-GUG, 5′-UUG or 5′-CUG, and 5′-AUA, 5′-ACG and 5′-CUG and have been shown to function in vivo. Thus, the terms “translation initiation codon” and “start codon” encompasses in other embodiments, many codon sequences, even though the initiator amino acid in each instance is typically methionine (in eukaryotes) or formylmethionine (in prokaryotes). In another embodiment, “start codon” and “translation initiation codon” refer to the codon or codons that are used in vivo to initiate translation of an mRNA molecule transcribed from a gene encoding HSV-LAT, regardless of the sequence(s) of such codons.

In certain embodiments, a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer in one embodiment, to a portion of such a mRNA or gene that encompasses from about 25 to about 50 contiguous nucleotides in either direction (i.e., 5′ or 3′) from a translation initiation codon. In another embodiment, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” refers in one embodiment to the region between the translation initiation codon and the translation termination codon, is a region which may be targeted effectively. Other target regions include in other embodiments, the 5′ untranslated region (5′UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises in one embodiment, an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region is a preferred target region in one embodiment.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be target regions in one embodiment, and are particularly useful in situations where aberrant splicing is implicated in disease, or where an overproduction of a particular mRNA splice product is implicated in disease in other embodiment, such as symptoms associated with HSV infection. Aberrant fusion junctions due to rearrangements or deletions are also preferred targets. In one embodiment, introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect. In one embodiment, the term “hybridization” refers to hydrogen bonding, which may be Watson-Crick, Hoogsteen or reversed Hoogsteen hydrogen bonding, between complementary nucleoside or nucleotide bases. In one embodiment, adenine and thymine are complementary nucleotide bases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, or in the case of in vitro assays, under conditions in which the assays are performed.

Antisense compounds are used in one embodiment, as research reagents and diagnostics. In another embodiment, antisense oligonucleotides, which are able to inhibit gene function, such as the HSV-LAT gene, with extreme specificity, are used by those of ordinary skill to elucidate the function of particular genes. Antisense compounds are used in another embodiment, to distinguish between functions of various members of a biological pathway. Antisense modulation has, in one embodiment of the agents described in the methods and compositions described herein, been harnessed for research use.

In one embodiment, the specificity and sensitivity of antisense agents described herein, is also harnessed for therapeutic uses. Antisense oligonucleotides are employed in one embodiment, as therapeutic moieties in the treatment of disease states in animals and man, such as, in another embodiment, those associated with HSV infection. In one embodiment, antisense oligonucleotides are safely and effectively administered to humans. In one embodiment oligonucleotides are useful therapeutic modalities that can be configured to be useful in treatment regimes of cells, tissues and animals, especially humans. In one embodiment, the term “oligonucleotide” refers to an oligomer or polymer of ribonucleic acid (RNA) or deoxyribonucleic acid (DNA) or mimetics thereof. This tern includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

In one embodiment, the oligonucleotides used in the methods and compositions described herein, are synthetic peptide nucleic acids (PNAs) which interact with the nucleotide sequence encoding mirLAT, comprising SEQ. ID No.'s 1, 5 and 6 in a sequence-specific manner and silence function of HSV-LAT. In another embodiment, the oligonucleotides used in the methods and compositions described herein, are locked nucleic acid (LNA), which interact with the nucleotide sequence encoding mirLAT; comprising SEQ. ID No.'s 1, 5 and 6, forming a LNA/DNA co-polymer, in a sequence-specific manner and substantially silence the function of HSV-LAT.

In one embodiment, the term “locked nucleic acid” (LNA) refers to a synthetic nucleic acid analogue, incorporating “internally bridged” nucleoside analogues. Synthesis of LNA, and properties thereof, have been described by a number of authors: Nielsen et al, (1997 J. Chem. Soc. Perkin Trans. 1, 3423); Koshkin et al, (1998 Tetrahedron Letters 39, 4381); Singh & Wengel (1998 Chem. Commun. 1247); and Singh et al, (1998 Chem. Commun. 455). As with PNA, LNA exhibits greater thermal stability when paired with DNA, than do conventional DNA/DNA heteroduplexes. In one embodiment, LNA can be joined to DNA molecules by conventional techniques. Therefore, in one embodiment, LNA is to be preferred over PNA, for use in the agents of the methods and compositions described herein.

In one embodiment, the target specific regions of the agent that is able to inhibit gene function, such as the HSV-LAT gene, may comprise LNA and/or PNA and the arm region comprise DNA, with the agent further comprising a destabilizing moiety.

In another embodiment, the agent capable of inhibiting expression or function of Men1 gene, or its encoded protein is an agPNA. In another embodiment, this antibody is referred to as antigenic PNA.

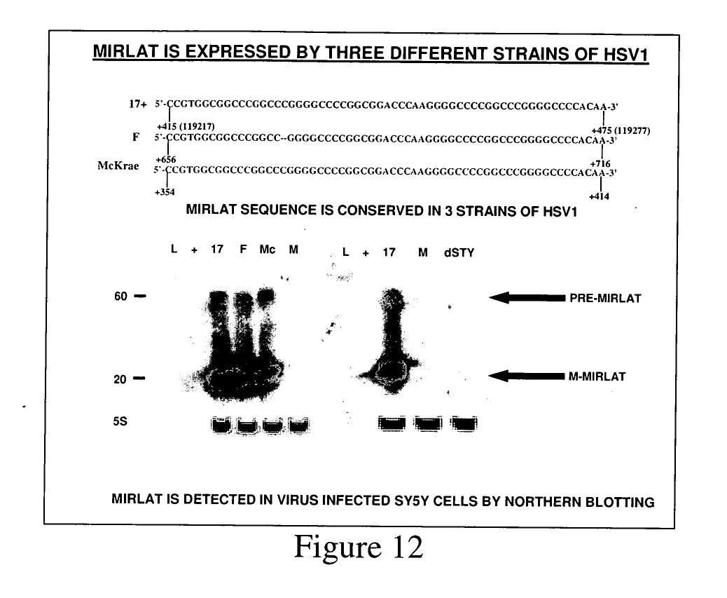

In another embodiment, mirLAT inhibits apoptosis by modulation of TGF-β signaling (see FIG. 12). Thus, in another embodiment, HSV uses the RNAi pathway to regulate host cell apoptosis to ensure survival of infected neurons and maintenance of latent infection. Such a mechanism circumvents the need for the expression of a viral protein during the latent infection and thus help the virus to evade immune detection.

The transforming growth factor-.beta.(TGF-β) family of proteins consists of a number of related, but functionally distinct, proteins. Members of this group mediate a wide range of biological processes in vertebrates and invertebrates, includings regulation of cell proliferation, differentiation, recognition, and death, and thus play a major role in developmental processes, tissue recycling, and repair. Members of the TGF-β family of proteins initiate cell signaling by binding to heteromeric receptor complexes of type I (T β RI) and type II (T β RII) serine/threonine kinase receptors. Activation of this heteromeric receptor complex occurs when TGF-β binds to T β RII, which then recruits and phosphorylates T β RI. Activated T β RI then propagates the signal to downstream targets. One member of the TGF-β family of proteins, TGF-β1, is a multifunctional cytokine with both growth promoting and inhibiting activities. Also, constitutively active type I receptors appear to signal biological responses in the absence of ligand and receptor II (RII), which indicates the role of the type I receptor (RI) as the downstream or the essential transducing element.

In one embodiment, TGF-β binding causes receptor serine/threonine kinases of the TGF-β receptor subfamily to phosphorylate and activate receptor-regulated Smads (R-Smads), Smad2 and Smad3, and/or initiate non-Smad signaling through activation of mitogen-activated protein kinases (MAPKs), phosphatidylinositol 3-kinase, and other mediators. Activated R-Smads heterooligomerize with the common partner (CO)-Smad4 before translocation to the nucleus, where they regulate gene expression. R-Smads and CO-Smads contain highly conserved Mad homology 1 (MH1) and MH2 domains, connected by a linker region. The MH1 domain of Smad3 mediates direct interaction of Smad3 with conserved DNA Smad-binding elements (SBEs). These complexes are subsequently translocated into the nucleus and act as TGF-β1-sensitive transcriptional coactivators or corepressors by interacting with a variety of transcription factors. In one embodiment, TGF-β1 elicits apoptotic cell death in a variety of cell types. Moreover, TGF-β1-induced apoptosis plays important roles in the selective elimination of damaged or abnormal cells from various normal tissues. In one embodiment, inhibition of expression of TGF-b1, inhibits apoptosis of HSV-infected cells, thereby maintaining survival of the virus and its latency. In one embodiment, the methods and compositions described herein, comprise the administration or the composition of a second agent, capable of upregulating the expression of TGF-β1, SMAD-3 or both.

In one embodiment, the term “treatment”, or “treating” refers to any process, action, application, therapy, or the like, wherein a subject, including a human being, is subjected to medical aid with the object of improving the subject's condition, directly or indirectly. The term “treating” refers also to reducing incidence, or alleviating symptoms, eliminating recurrence, preventing recurrence, preventing incidence, improving symptoms, improving prognosis or combination thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent infection. The alleviation of a condition that results in a more serious condition is encompassed by this term. Therefore, in one embodiment, provided herein a method of treating HSV infection, in a human subject, comprising the step of contacting a peripheral neuron cell of said subject with an effective amount of an agent capable of inhibiting the function of a miRNA of the HSV-LAT gene or its encoded proteins, whereby the inhibition of expression or function of the miRNA of the HSV-LAT gene or its encoded proteins results in apoptosis in said peripheral neuron cell, thereby reducing recurdescence of HSV infection.

In one embodiment, the term “up-regulate” refers to the expression of the gene, or level of RNAs or equivalent RNAs encoding one or more protein subunits, or activity of one or more protein subunits, such as TGF-β1 or SMAD3 or both, is greater than that observed in the absence of the agents described in the methods, compositions and vaccines described herein. For example, the expression of a gene, such as TGF-β1 or SMAD3 or both, can be increased in order to treat, prevent, ameliorate, or modulate a pathological condition caused or exacerbated by an absence or low level of gene expression.

In one embodiment, the agents described hereinabove are used in the compositions described herein. In another embodiment, the compositions described herein are used in the methods described herein.

In one embodiment, provided herein is a composition for the treatment of Herpes Simplex Virus infection in a subject, comprising an agent capable of inhibiting the function of a HSV latency-associated transcript (LAT) gene.

In another embodiment, provided herein is a method for inhibiting apoptosis of neuronal cells in a subject, comprising contacting said neuronal cells with a miRNA of HSV-LAT gene, wherein said miRNA modulates the TGF-b pathway, thereby inhibiting apoptosis of said neuronal cells. In one embodiment, the miRNA modulates the TGF-b pathway by down-regulates the expression of TGF-β1, SMAD-3 or both.

In one embodiment, provided herein is a method of screening for therapeutic agents for the treatment of HSV infection in a subject, comprising the step of: contacting a neuronal cell of said subject with the candidate therapeutic agent; and analyzing for the function of HSV-LAT gene or its encoded proteins in said contacted cell, wherein inhibition of the function of HSV-LAT gene or its encoded proteins in said neuronal cell indicates the candidate therapeutic agent is effective in treating HSV infection.

In one embodiment, The term ” therapeutic agents” or “candidate therapeutic agents” refers to a compound that possesses or has been modified to possess a reactive group that is capable of forming a covalent, or non-covalent bond with a complimentary or compatible reactive group on a target, such as the HSV mirLAT comprising SEQ ID No.'s 1, 5 and 6. The reactive group on either the therapeutic agents candidate or the target can be masked with, for example, a protecting group. In one embodiment, the term “therapeutic agents” refers to an entity that possesses a measurable binding affinity for the target. In another embodiment, a therapeutic agents is said to have a measurable affinity if it binds to the target with a K_{d} or a Kᵢ of less than about 100 mM. In certain embodiments, the ligand is not a peptide and is a small molecule. A ligand is a small molecule if it is less than about 2000 daltons in size, usually less than about 1500 daltons in size. In another embodiment, the small molecule therapeutic agents is less than about 1000 daltons in size, usually less than about 750 daltons in size, and more usually less than about 500 daltons in size.

Initially a potential therapeutic agent could be obtained by screening a random peptide library produced by recombinant bacteriophage in one embodiment, [Scott and Smith, Science, 249:386-390 (1990); Cwirla et al., Proc. Natl. Acad. Sci., 87:6378-6382 (1990); Devlin et al., Science, 249:404-406 (1990)] or a chemical library. An agent thus selected in another embodiment, could then be be systematically modified by computer modeling programs until one or more promising potential drugs are identified. Such analysis has been shown to be effective in the development of HIV protease inhibitors [Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1: 109-128 (1993)].

In one embodiment, computer modeling allows the selection of a finite number of rational chemical modifications, as opposed to the countless number of essentially random chemical modifications that could be made, any one of which might lead to a useful ligand. Each chemical modification requires additional chemical steps, which while being reasonable for the synthesis of a finite number of compounds, may become overwhelming if all possible modifications are needed to be synthesized. Thus through the use of a three-dimensional structural analysis and computer modeling, a large number of these compounds can be rapidly screened on the computer monitor screen, and a few likely candidates can be determined without the laborious synthesis of numerous compounds.

In one embodiment, inhibiting the function of a HSV latency-associated transcript (LAT) gene or its encoded proteins, using the agents described herein, comprises lowering the level of a protein or a nucleic acid regulating the function of said latency-associated transcript (LAT) gene, or its encoded proteins. In one embodiment, the nucleic acid whose level is reduced is the nucleic acid represented by SEQ. ID No.'s 1, 5 and 6, which in another embodiment comprises a miRNA of HSV-LAT.

In one embodiment, the candidate therapeutic agent identified in the screening methods described herein is a siRNA, polyamides, triple-helix-forming agents, antisense RNA, synthetic peptide nucleic acids (PNAs), agRNA, LNA/DNA copolymers, small molecule chemical compounds, or a combination thereof, specific for the nucleic acid represented by SEQ. ID No.'s 1, 5 and 6, which in another embodiment comprises a miRNA of HSV-LAT.

In one embodiment, the agents used in the methods and compositions described herein, and those agents identified using the screening methods described herein, are used in the vaccines described herein.

In one embodiment, provided herein is a vaccine for treating, preventing or ameliorating a subject against herpes simplex virus infection or recrudescence, comprising a pharmaceutically acceptable carrier and an effective amount of an agent capable of inhibiting the function of a HSV-latency-associated transcript (LAT) gene in said subject, thereby allowing for apoptosis of infected neurons. In one embodiment, the vaccines described herein, further comprise another agent, capable of killing HSV, such as ACYCLOVIR™ in another embodiment.

In one embodiment, the vaccines described herein may contain other agents specific against several other regions of the HSV genome relating to viral neurovirulence. These regions include those containing the thymidine kinase gene, the DNA polymerase gene, sequences within the internal repeats, and sequences between map units (“mu”) 0.25 and 0.53.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

Materials and Methods:

Cells and Virus

SY5Y cells were maintained in RPMI medium with 10% fetal calf serum and antibiotics. HeLa cells were grown in DMEM medium supplemented with 5% fetal calf serum and antibiotics. Plasmids having the PstMlu fragment of LAT and its deletion mutant ΔSty have been described. The wild type strain of HSV-1 (17+), and the mutant virus (ΔSty) have been described. Apoptosis assay

24 hours post-transfection cells were treated with cisplatin (10 □M) or etoposide (1-100 μM), followed by staining with AnnexinV FITC (BD Biosciences). Cells showing FITC staining, loss of cell volume, loss of refractility, and membrane blebbing were scored as apoptotic. At least 500 cells were counted in each well.

RNA Isolation, RT-PCR, Northern Blotting and Western Analysis

RNA was isolated using Trizol reagent (Invitrogen). RNA was reverse transcribed using Superscript II RT enzyme (Invitrogen). Northern blot analysis of 50-60 μg total RNA was done as described previously. Western blot analysis was performed on whole cell lysates using Dicer polyclonal antibody and β-actin antibody (Amersham). SMAD-3 and TGF-β receptor I antibodies were from Abcam.

Reporter Assays

Cells were co-transfected with pCDNA or PstMlu (1 μg) and the respective reporter constructs (500 ng) along with pSV-βgal (10 ng). For induction of TGF-p signaling, 24 h following transfection the cells were treated with 10 ng/ml of recombinant TGF-β1 protein (R&D systems). Reporter activity was measured using a appropriate assay kits (promega).

miRNA Cloning

RNAMOT was used to search for RNA hairpin. The detected duplexes were further analyzed by MFOLD. Targets of mirLAT were identified using the software miRanda. Statistical analysis was performed using JMPIN 4 software. One way ANOVA analysis was performed at an alpha level of 0.01-0.05 to compare all the variants in a data set.

Cells and Virus

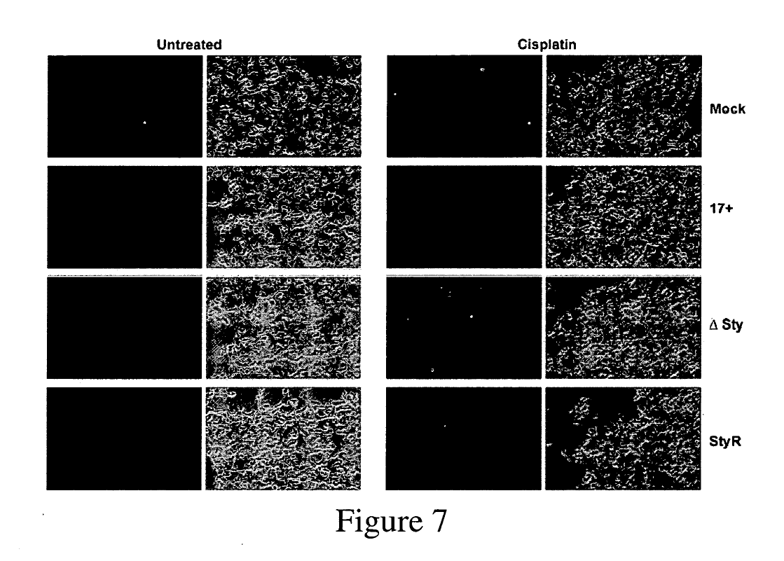

SY5Y cells were maintained in RPMI medium with 10% fetal calf serum and antibiotics. HeLa cells were grown in DMEM medium supplemented with 5% fetal calf serum and antibiotics. Plasmids having the PstMlu fragment of LAT and its deletion mutant ΔSty have been described. GFP-shRNA pSilencer plasmid was a kind gift from Dr Kuan-Teh Jeang, NIH, USA. The SBE4-Luc construct was a kind gift from Dr Anita Roberts, NIH, USA. The pGL3-control and pSV-βgal plasmids were obtained from Promega. The pRL-TK vector was obtained from Dr Zissimos Mourelatos, University of Pennsylvannia, USA. The full length 3′ UTR of TGF-β (2041-2341 bp of GI:63025221) and SMAD-3 (1571-2560 bp of GI:52352808) were PCR amplified from reverse transcribed cDNA of SY5Y cells using appropriate primers and cloned into the XbaI-NotI sites at the 3′ end of the renilla luciferase gene in the pRL-TK vector. The sequence of the oligonucleotides corresponding to the wild type and mutant target sites are described in FIG. 7. These annealed duplex oligos were cloned into the NdeI-XhoI site at the 3′ end of the renilla luciferase gene in the vector PRL-TK to obtain the wild type and mutant constructs. All constructs were confirmed by sequencing. The wild type strain of HSV-1 (17+), and the mutant virus (ΔSty) have been described. ΔSty rescued virus (StyR) was synthesized by co-transfecting mutant virus genomic DNA (ΔSty) and plasmid which contained the ΔSty region as well as flanking sequences into CV-1 cells. Recombinants were scored for reversion of deletion in ΔSty resulting in virus which is genetically similar to wild type virus. The genomic structure of the rescuant, called StyR was verified by Southern blot experiments. Infection was done at MOI of 1-5 for 16 hours at 37° C.⁵. Transfections were done using Lipofectamine 2000 (Invitrogen) according to manufacturer's instructions. The Dicer SiRNA used (Dharmacon) has been described. The sequence of the mirLAT dsRNA oligonucleotide used was 5′-GUGGCGGCCCGGCCCGGGGCCTT-3′ (SEQ ID NO. 1, Dharmacon). The control miRNA used was a ˜20 nt scrambled non-targeting RNA duplex (Dharmacon). A 2′O-methyl antisense oligonucleotide having a sequence complementary to mirLAT and a control 2′-O-methyl oligo of the same length was obtained from Integrated DNA technologies (IDT). The siRNAs directed against TGF-β (CCAACUAUUGCUUCAGCUCTT) (SEQ ID NO. 2) and SMAD-3 (GCACAUAAUAACUUGGACCTT) (SEQ ID NO. 3) were obtained from Ambion.

Apoptosis Assay

24 hours post-transfection cells were treated with cisplatin (10 μM) or etoposide (1-00 μM), followed by staining with AnnexinV FITC (BD Biosciences). Cells showing FITC staining, loss of cell volume, loss of refractility, and membrane blebbing were scored as apoptotic. At least 500 cells were counted in each well. The data represent mean±S.D. from at least three independent experiments.

RNA Isolation, RT-PCR, Northern Blotting and Western Analysis

RNA was isolated using Trizol reagent according to the manufacturer's instruction. RNA was reverse transcribed using Superscript II RT enzyme (Invitrogen). Dicer, TGF-β 1, SMAD-3, TGF-β receptor I and GAPDH cDNA were amplified using gene specific primers. Northern blot analysis of 50-60 μg total RNA was done. Radioactive probes were generated using Starfire oligonucleotide labeling system (IDT). Western blot analysis was performed on whole cell lysates using Dicer polyclonal antibody, a kind gift from Dr Ramin Shiekhattar, Wistar Institute, Philadelphia, USA and β-actin antibody (Amersham). SMAD-3 and TGF-β receptor I antibodies were from Abcam. Elispot assay for determining the number of latent TGF-β producing cells was done using a Human latent TGF-β1 Elispot kit from R&D systems. Synthetic RNA molecule corresponding to the mirLAT pre-miRNA (Dhramacon) was radiolabelled by polynucleotide kinase reaction (Invitrogen), column purified and incubated with 2 U Recombinant Dicer enzyme (Stratagene) at 37° C. for 16 hours and analyzed by gel electrophoresis and exposure to a phosphor imager screen.

Reporter Assays

Cells were co-transfected with pCDNA or PstMlu (1 μg) and the respective reporter constructs (500 ng) along with pSV-βgal (100 ng). For induction of TGF-β signaling, 24 h following transfection the cells were treated with 10 ng/ml of recombinant TGF-β 1 protein (R&D systems) for 12-16 hours at 37° C. in serum free media. Cells were lysed using the reporter lysis buffer (promega). Luciferase activity was measured using a renilla or firefly Luciferase assay kit (promega). β-galactosidase activity was determined in all lysates (promega) according to manufacturer's instructions. The Luciferase activity in all samples was normalized to the amount of β-galactosidase activity.

miRNA Cloning

Small RNA molecules were cloned from PstMlu transfected cells. Briefly, 600 μg RNA was run on a 15% polyacrylamide-urea gel along with radiolabelled RNA markers of 18 and 24 mer sizes. RNA of 18-24 bp size range was purified from the gel and ligated sequentially to the 3′ miRNA cloning linker (IDT) and adenylated 5′ acceptor (ATCGTaggcaccugaaa) (SEQ ID NO. 4) oligos. The ligation products were converted to cDNA using superscript II RT enzyme (Invitrogen) and was amplified by PCR. The amplified cDNA library was digested with BanI and ligated to concatemerize. Concatemers ranging in size from 100-1000 bp were gel purified and cloned into PCR4-TOPO vector using a PCR TOPO TA cloning kit (Invitrogen) according to the manufacturer's protocols. The colonies obtained were screened by colony hybridization analysis using the 372 nt Sty-Sty fragment of the HSV-LAT gene as probe. Positive clones were sequenced using M 13 forward and reverse primers and sequences were analyzed using MacVector software.

Computational Methods

RNAMOT was used to search for RNA hairpin structures in progressive 20 bp stretches using the criteria that the minimal duplex stem is 18 bp with <3 mismatches. The detected duplexes were further analyzed by MFOLD using the following parameters: folding temperature of 37° C., 5% sub-optimality, maximum interior bulge or loop size of 15 nucleotides and maximum asymmetry of an interior bulge or loop of 5 nucleotides. Targets of mirLAT were identified using the software miRanda. Statistical analysis was performed using the JMPIN 4 software. One way ANOVA analysis was performed at an alpha level of 0.01-0.05 to compare all the variants in a data set.

Calculation of Prevalence of mirLAT in Infected Cells.

Usually 100 mg RNA per lane is loaded on gel to detect mirLAT by northern blot. This RNA is obtained from approximately 10⁶ cells, routinely 70% of cells are transfected and express mirLAT (i.e 7×10⁵ cells).

Band intensity of mirLAT detected matches with band intensity obtained from 10⁻¹⁵ mols of synthetic mirLAT, that is approximately 10⁸ molecules per lane.

Hence, mirLAT is present at a concentration of 1000 000 00/7×1000 00

### Example 1

### HSV-1 LAT Gene Inhibits Apoptosis

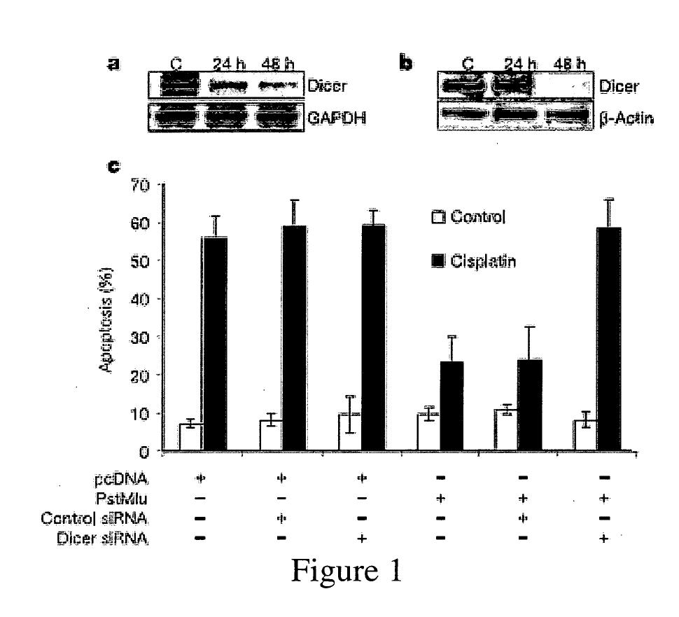

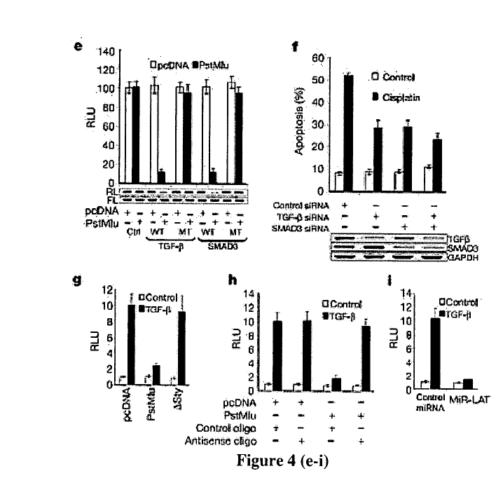

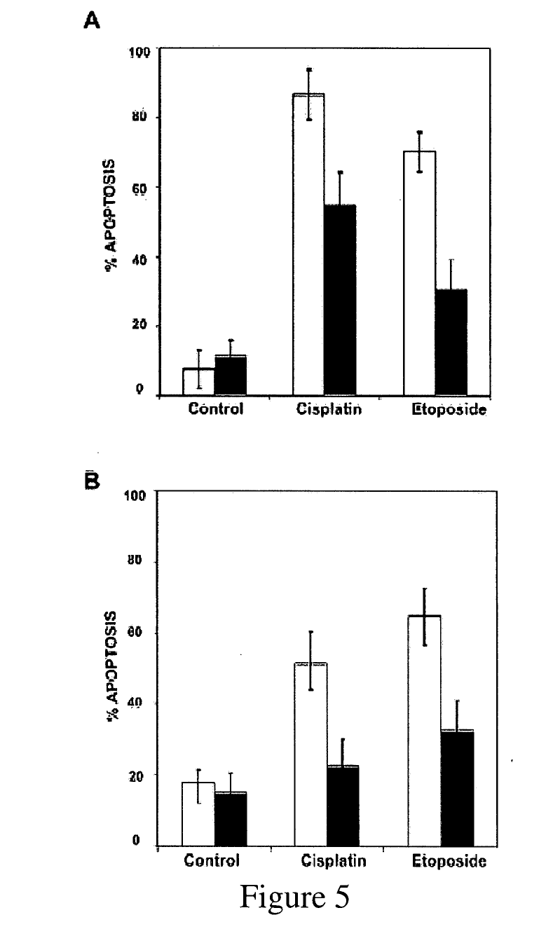

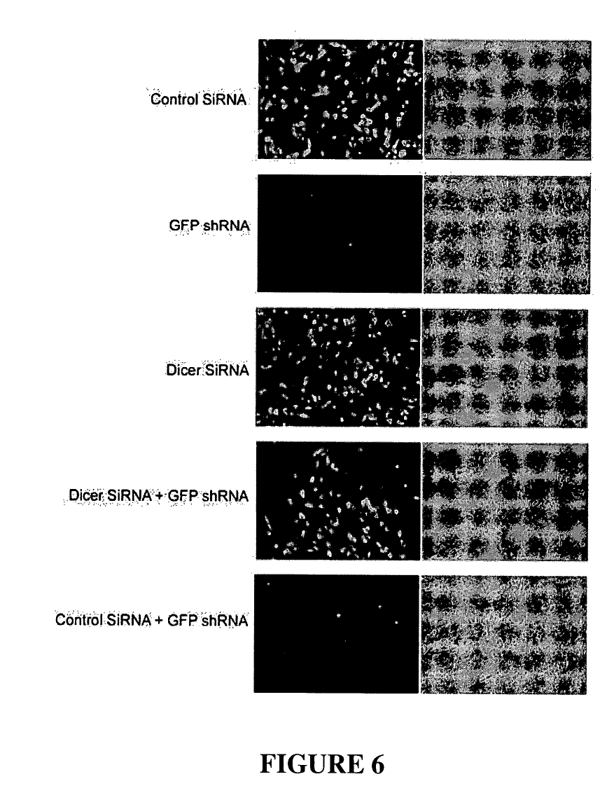

The anti-apoptotic function of LAT was first confirmed. As expected a 2.9 kb fragment of the HSV-1 LAT gene (pcDNA-PstMlu) reduced susceptibility to stress-induced apoptosis in SY5Y and HeLa cells (FIG. 5). Though it has been shown that LAT promotes neuronal survival following HSV infection by reducing apoptosis, the mechanism by which it regulates this process is still not understood. It was hypothesized that the HSV-1 LAT gene exerts its anti-apoptotic effect by encoding a miRNA. To test this hypothesis, the function of the RNAse III enzyme Dicer was attenuated by using specific siRNA (FIG. 1a,b) and its effect on LAT-mediated resistance to apoptosis was evaluated. Dicer siRNA abrogated the ability of a short hairpin RNA (shRNA) directed against GFP (GFP-shRNA) to block GFP expression in (FIG. 6). Dicer siRNA significantly inhibited the anti-apoptotic effect of LAT as compared to cells transfected with a control non-specific siRNA (FIG. 1c)

### Example 2

### LAT Gene of HSV Codes for a miRNA

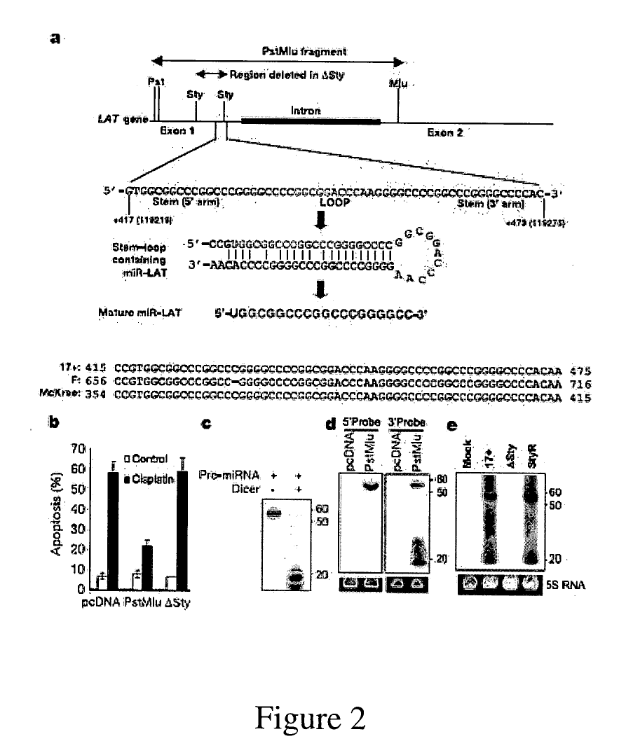

Computational analysis using RNAMOT and MFOLD revealed the presence of a sequence within exon-1 of HSV-LAT containing a hairpin loop reminiscent of precursor miRNAs (FIG. 2a). Next the small RNAs from SY5Y cells transfected with pcDNA-PstMlu were cloned. Colony hybridization with the Sty-Sty region of LAT revealed that 7% of the clones contained mirLAT sequences. Sequencing of positive clones confirmed the predicted sequence of mirLAT shown in FIG. 2a. The mirLAT was cloned multiple times. No other HSV miRNA was present in the sequenced clones. The sequences present in the HSV LAT region encoding for mirLAT microRNA are conserved in 17+, F and McKrae strains of HSV-1 (FIG. 2a, FIG. 12). Transfection of cells with a LAT deletion construct (ΔSty), in which a 372 nt fragment, encompassing the predicted mature miRNA (FIG. 2a, FIG. 12), has been deleted, showed significant reduction in protection from cisplatin-induced apoptosis (FIG. 2b).

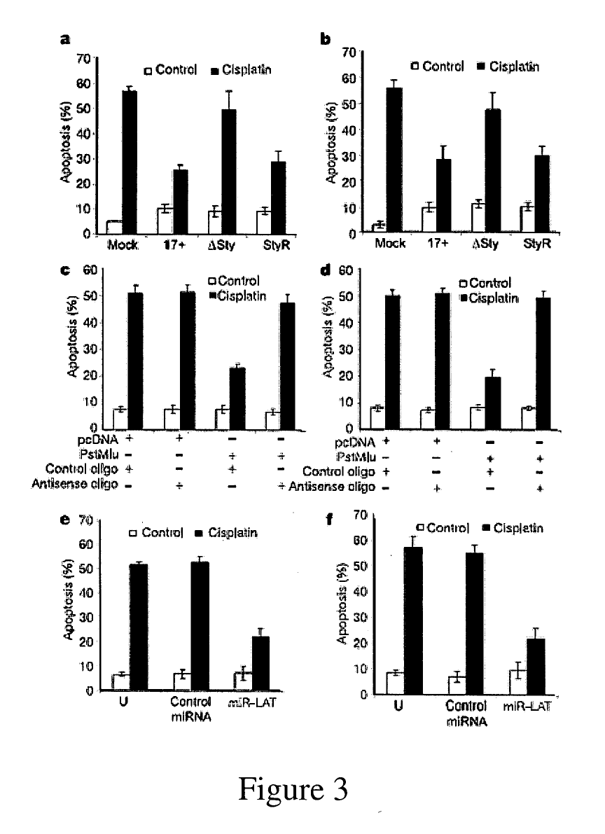

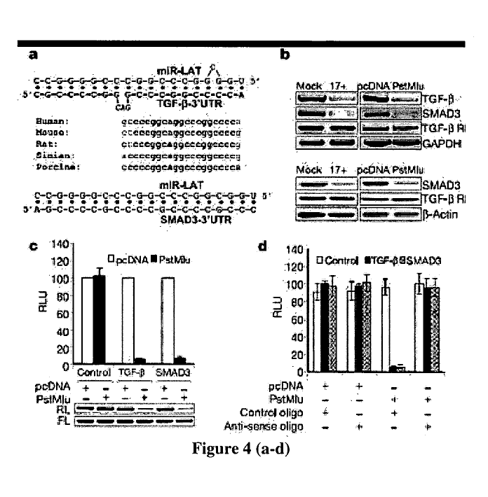

A synthetic RNA oligonucleotide corresponding to the pre-mirLAT was processed in-vitro by a recombinant Dicer enzyme to yield a ˜20 nt product (FIG. 2c). Next Northern blot analysis was performed, to evaluate the expression mirLAT in LAT-expressing cells. Only one of the strands of the double stranded hairpin precursor is energetically favored to enter the RISC complex and accumulate in the cell as the mature miRNA. Therefore two probes, the 5′ probe comprising of the 5′ arm of the hairpin, and the 3′ probe comprising of the 3′ arm of the hairpin were used. The 3′ probe revealed a ˜60 nt and a ˜20 nt band (FIG. 2d), corresponding to a pre-miRNA and a mature miRNA, respectively. The 5′ probe did not show any mature miRNA (FIG. 2d). However, the ˜60 nt pre-miRNA band was detected by the 5′ probe. This suggests that only the 5′ arm of the pre-miRNA hairpin constitutes the mature mirLAT. Cells transfected with the plasmid pcDNA-ΔSty, a deletion mutant of LAT, were not protected from apoptosis (FIG. 2b), and did not show any pre- or mature miRNA in northern blot analysis. Next the generation of mirLAT was determined in virus-infected cells. For this purpose the wild type virus (17+), mutant virus ΔSty) with a 372 nt deletion in exon-1 (position +76 to +447) of the LAT gene, and ΔSty rescued virus (StyR) were used. The mutation did not affect the ability of the virus to establish infection in tissue culture and the expression of other viral genes. As shown in FIG. 2e, ˜20 nt mature mirLAT was detected in the cells infected with the wild type and StyR virus. No mature mirLAT was found in the cells infected with the mutant virus (FIG. 2e). Furthermore, the ΔSty virus was less efficient in protecting cells from stress-induced apoptosis as compared to the wild type 17+ and the rescued StyR virus (FIG. 3a-b; FIG. 7).

### Example 3

### The LAT Region of HSV 17+ Protects Cells from Apoptosis

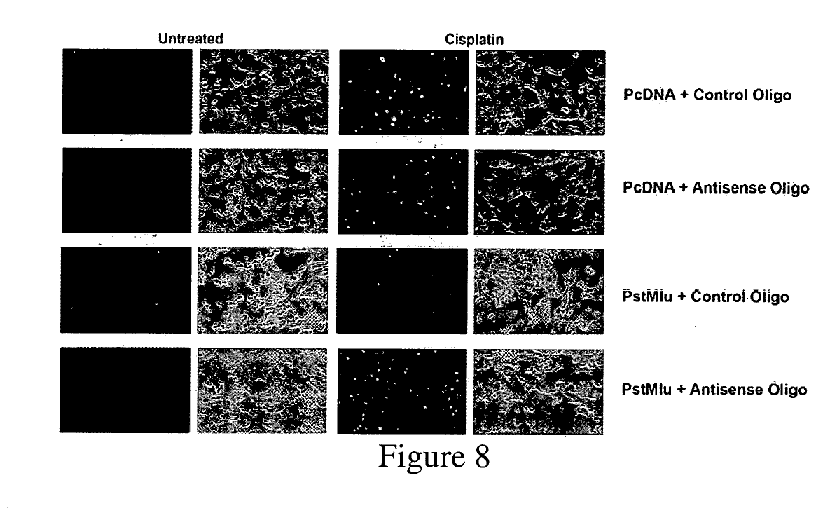

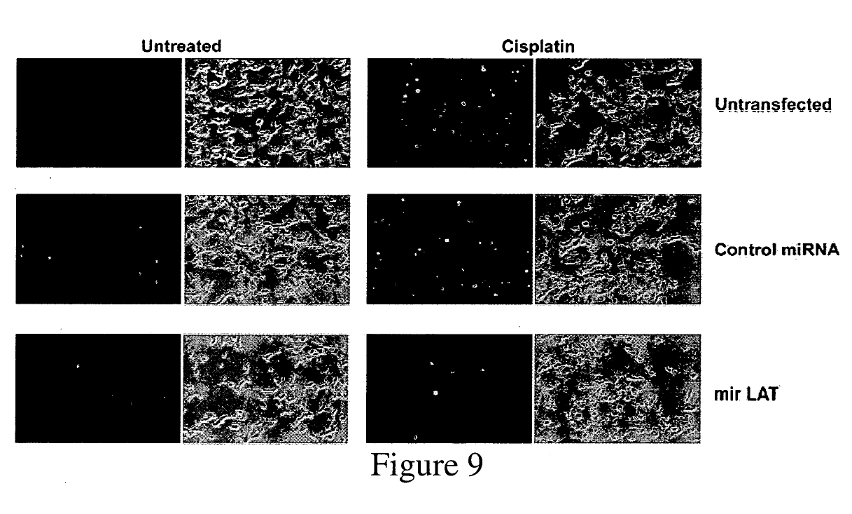

To determine the role of mirLAT in providing resistance to apoptosis 2′-O-methyl antisense oligonucleotides specific for mirLAT was used. As shown in FIG. 3c-d, co-transfection of the 2′-O-methyl antisense oligonucleotide with the LAT fragment (pcDNA-PstMlu) significantly attenuated its anti-apoptotic effect, whereas the control 2′-O-methyl oligonucleotide had no such effect. Both antisense and control 2′-O-methyl oligonucleotides did not show any effect on apoptosis when cotransfected with control (PcDNA) plasmid (FIG. 3c-d; Supplementary FIG. 8). To further elucidate the anti-apoptotic function of mirLAT cells were transfected with a synthetic double stranded RNA oligonucleotide corresponding to the mature mirLAT. MirLAT caused significant reduction in apoptosis as compared to the control miRNA (FIG. 3e-f; Supplementary FIG. 9)

### Example 4

### mirLAT Modulates of TGF-β Signaling

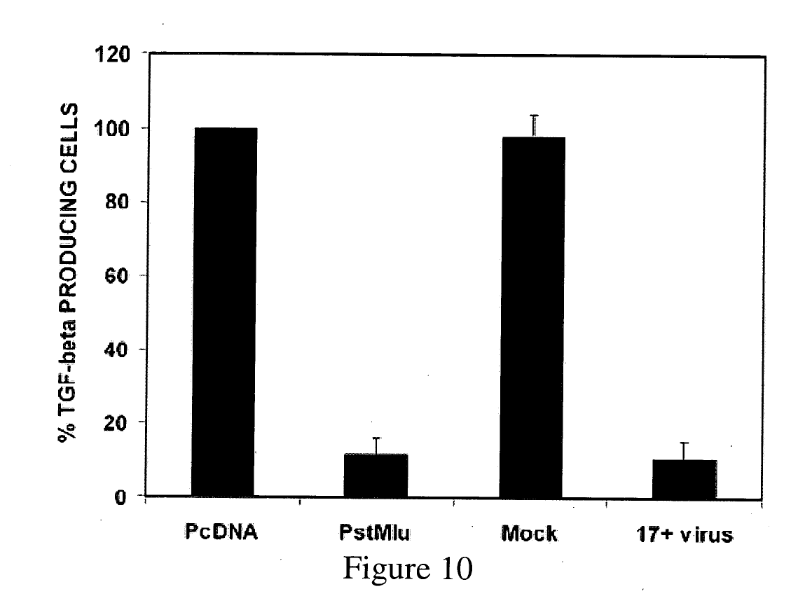

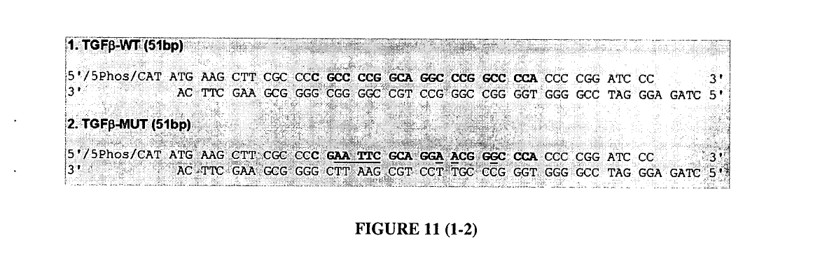

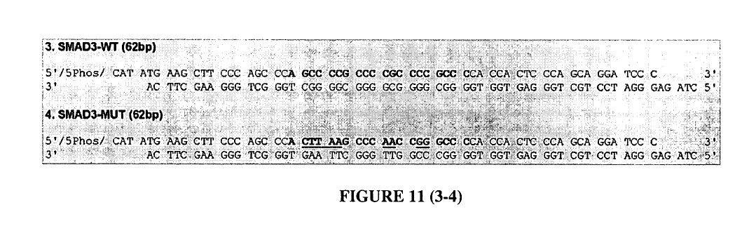

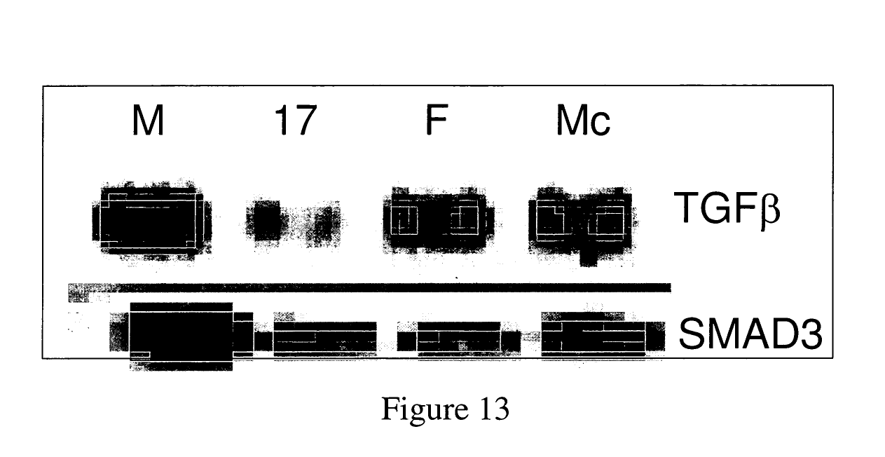

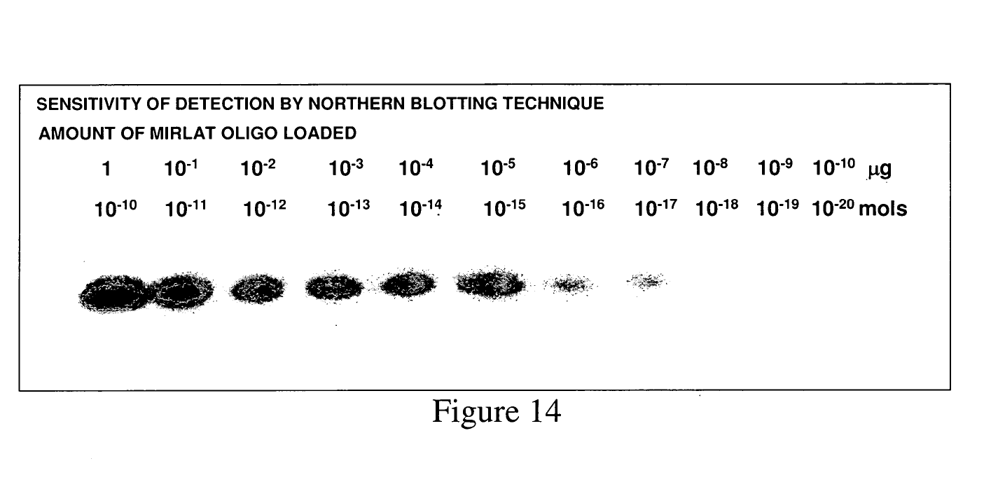

To gain insight into the mechanism by which mirLAT regulates apoptosis, targets of mirLAT were sought to be identified. Computational analysis using miRanda identified, Transforming growth factor-β 1 (TGF-β) and SMAD-3 as targets of mirLAT, both of which are functionally related in TGF-β signaling. TGF-β is a potent inhibitor of cell growth and an inducer of apoptosis. SMAD-2 and SMAD-3 are phosphorylated by the activated TGF-β receptor, form complexes with SMAD-4, and together accumulate in the nucleus to regulate transcription of target genes that play important roles in diverse cellular processes. TGF-β-induced cell death is associated with changes in the expression, localization and activation of both pro- and anti-apoptotic members of the Bcl2 family, as well as activation of caspases. The 3′UTR of TGF-β and SMAD-3 contain sequences with partial homology to mirLAT (FIG. 4a). The mirLAT response element in the 3′ UTR of TGF-β was found to be evolutionarily conserved (FIG. 4a). Given that miRNA's function in RNA silencing pathways either by targeting messenger RNAs (mRNA) for degradation or by repressing translation, we determined the effect of HSV-1 LAT on mRNA and protein levels of TGF-β and SMAD-3. Substantial decrease was observed in the mRNA levels of TGF-β and SMAD-3 in cells transfected with pcDNA-PstMlu or infected with wild type 17+ virus (FIG. 4b) which was accompanied by a concomitant decrease in endogenous SMAD-3 (FIG. 4b) and TGF-β protein (FIG. 10). a significant decrease was observed in the activity of the renilla luciferase reporter gene fused to the full length 3′UTRs of TGF-β or SMAD-3 in LAT-expressing SY5Y cells, which was accompanied by a decrease in renilla luciferase mRNA (FIG. 4c). There was no effect on the activity and mRNA levels of a renilla luciferase reporter gene lacking the mirLAT response elements (FIG. 4c). Further, LAT-expression had no effect on mRNA levels of firefly luciferase, used as a transfection control (FIG. 4c). LAT-expression had no affect on the activity of a renilla luciferase (RL) reporter construct having the 3′UTR of TGF-β or SMAD-3 in the presence of a 2′-O-methyl antisense oligonucleotide specific for mirLAT (FIG. 4d). Further to confirm that repression of TGF-β and SMAD-3 by mirLAT is via the predicted mirLAT-binding site in their 3′ UTRs (FIG. 4a, FIG. 13), double-stranded oligos were cloned, comprising the wild-type target site or a mutant target site that disrupts mirLAT binding, at the 3′ end of the renilla luciferase gene. These clones contained a single copy of either the wild-type or the mutated target site. The wild-type construct (TGF-β and SMAD-3) showed up to 90% reduction in luciferase activity, which was accompanied by a decrease in renilla luciferase mRNA (FIG. 4e). The mutant construct does not show any significant decrease in luciferase activity or mRNA levels of a renilla luciferase reporter gene (FIG. 4e). Thus, the proposed site (FIG. 4a) is the major mirLAT binding site in these target genes.

Next the expression of TGF-β and SMAD-3 was inhibited using siRNAs that specifically target TGF-β and SMAD-3 (FIG. 4f). Down-regulation of TGF-β and SMAD-3 independently of mirLAT also confers resistance to apoptosis (FIG. 4f), whereas a control scrambled siRNA did not show any such effect (FIG. 4f). This suggests that the inhibition of the TGF-β pathway by mirLAT is sufficient for its anti-apoptotic effect. See also FIG. 13.

Further the effect of mirLAT on TGF-β/SMAD-dependent transcription was analyzed, using SBE4-Luc, a TGF-β responsive reporter gene construct that contains four copies of the SMAD-binding element (SBE). As shown in FIG. 4g, pcDNA-PstMlu inhibited the TGF-β mediated induction of the SBE4-Luc construct. LAT deletion construct (ΔSty) had no effect on the induction of the SBE4-Luc reporter gene as compared to pcDNA (FIG. 4g). LAT-expression had no affect on TGF-β mediated induction of the SBE4-Luc reporter gene in the presence of 2′-O-methyl antisense oligonucleotides specific for mirLAT (FIG. 4h). To further elucidate the role of mirLAT in TGF-β signaling SY5Y cells were co-transfected with synthetic double stranded RNA oligonucleotide corresponding to the mature mirLAT along with the SBE4-Luc reporter gene. As shown in FIG. 4i, mirLAT caused a significant reduction in TGF-β mediated induction of the SBE4-Luc reporter gene activity as compared to a control (scrambled) double stranded RNA oligonucleotide.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method of treating Herpes Simplex Virus (HSV) infection in a subject, comprising administering to said subject an agent capable of inhibiting the function of a HSV-latency-associated transcript (LAT) gene in said subject, whereby said latency-associated gene inhibits apoptosis of infected neurons.

2. The method of claim 1, wherein inhibiting the function of a HSV latency-associated transcript (LAT) gene or its encoded proteins, comprises lowering the level of a protein or a nucleic acid regulating the function of said latency-associated transcript (LAT) gene, or its encoded proteins.

3. The method of claim 2, wherein said nucleic acid regulating the expression or function of said latency-associated transcript (LAT) gene, is a miRNA of said gene.

4. The method of claim 2, wherein the regulated function is the modulating of TGF-β pathway.

5. The method of claim 3, wherein said miRNA comprises the nucleotide sequence of SEQ ID NO. 1, SEQ ID No. 5 or SEQ ID NO. 6.

6. The method of claim 1, wherein said agent is a siRNA, polyamides, triple-helix-forming agents, antisense RNA, synthetic peptide nucleic acids (PNAs), agRNA, LNA/DNA copolymers, small molecule chemical compounds, or a combination thereof.

7. The method of claim 1, further comprising administering to said subject a second agent capable of up-regulating expression of TGF-β1, SMAD-3 or both.

8. The method of claim 1, wherein treating comprises reducing incidence, inhibiting or suppressing.

9. A composition for the treatment of Herpes Simplex Virus infection in a subject, comprising an agent capable of inhibiting the function of a HSV latency-associated transcript (LAT) gene.

10. The composition of claim 9, wherein said agent is a siRNA, polyamides, triple-helix-forming agents, antisense RNA, synthetic peptide nucleic acids (PNAs), agRNA, LNA/DNA copolymers, small molecule chemical compounds, or a combination thereof.

11. The composition of claim 9, wherein inhibiting the function of a HSV latency-associated transcript (LAT) gene or its encoded proteins, comprises lowering the level of a protein or a nucleic acid regulating the function of said latency-associated transcript (LAT) gene, or its encoded proteins.

12. The composition of claim 11, wherein said nucleic acid regulating the function of said HSV latency-associated transcript (LAT) gene, is a miRNA of said gene.

13. The composition of claim 12, wherein the regulated function is the modulating of TGF-β pathway.

14. The method of claim 12, wherein said miRNA comprises the nucleotide sequence of SEQ ID NO. 1, SEQ ID No. 5 or SEQ ID NO. 6.

15. The composition of claim 8, further comprising a second agent capable of up-regulating expression of TGF-β1, SMAD-3 or both.

16. A method for inhibiting apoptosis of neuronal cells in a subject, comprising contacting said neuronal cells with a miRNA of HSV-LAT gene, wherein said miRNA modulates the TGF-b pathway, thereby inhibiting apoptosis of said neuronal cells.

17. The method of claim 16, wherein said miRNA down-regulates the expression of TGF-β1, SMAD-3 or both.

18. A method of screening for therapeutic agents for the treatment of HSV infection in a subject, comprising the step of: contacting a neuronal cell of said subject with the candidate therapeutic agent; and analyzing for the function of HSV-LAT gene or its encoded proteins in said contacted cell, wherein inhibition of the function of HSV-LAT gene or its encoded proteins in said neuronal cell indicates the candidate therapeutic agent is effective in treating HSV infection.

19. The method of claim 18, wherein inhibiting the function of a HSV latency-associated transcript (LAT) gene or its encoded proteins, comprises lowering the level of a protein or a nucleic acid regulating the function of said latency-associated transcript (LAT) gene, or its encoded proteins.

20. The method of claim 19, wherein said nucleic acid regulating the function of said latency-associated transcript (LAT) gene, is a miRNA of said gene.

21. The method of claim 20, wherein the regulated function is the modulating of TGF-β pathway.

22. The method of claim 20, wherein said miRNA comprises the nucleotide sequence of SEQ ID NO. 1, SEQ ID No. 5 or SEQ ID NO. 6.

23. The method of claim 18, wherein treatment comprises reducing symptoms, inhibiting symptoms, or suppressing symptoms.

24. The method of claim 20, wherein said candidate therapeutic agent is a siRNA, polyamides, triple-helix-forming agents, antisense RNA, synthetic peptide nucleic acids (PNAs), agRNA, LNA/DNA copolymers, small molecule chemical compounds, or a combination thereof.

25. A vaccine for treating, preventing or ameliorating a subject against herpes simplex virus infection or recrudescence, comprising a pharmaceutically acceptable carrier and an effective amount of an agent capable of inhibiting the function of a HSV-latency-associated transcript (LAT) gene in said subject, thereby allowing for apoptosis of infected neurons.

26. The vaccine of claim 25, wherein inhibiting the function of a HSV latency-associated transcript (LAT) gene or its encoded proteins, comprises lowering the level of a protein or a nucleic acid regulating the function of said latency-associated transcript (LAT) gene, or its encoded proteins.

27. The vaccine of claim 26, wherein said nucleic acid regulating the expression or function of said latency-associated transcript (LAT) gene, is a miRNA of said gene.

28. The vaccine of claim 27, wherein the regulated function is the modulating of TGF-β pathway

29. The vaccine of claim 27, wherein said miRNA comprises the nucleotide sequence of SEQ ID NO. 1, SEQ ID No. 5 or SEQ ID NO. 6.

30. The vaccine of claim 21, further comprising ACYCLOVIR™.


SEQUENCE LISTING (6 sequences)

SEQ ID NO: 1 - DNA, 23 bp, HSV
guggcggcccggcccggggcctt

SEQ ID NO: 2 - DNA, 21 bp, Human
ccaacuauugcuucagcuctt

SEQ ID NO: 3 - DNA, 21 bp, Human
gcacauaauaacuuggacctt

SEQ ID NO: 4 - DNA, 17 bp, Human
atcgtaggcaccugaaa

SEQ ID NO: 5 - DNA, 57 bp, HSV
gtggcggcccggcccggggccccggcggacccaaggggccccggcccggggccccac

SEQ ID NO: 6 - DNA, 20 bp, HSV
uggcggcccggcccggggcc